(19) United States
(12) Patent Application Publication
(10) Pub. No.: US 2023/0405238 A1
(43) Pub. Date: Dec. 21, 2023

(54) SYSTEMS AND METHODS FOR DRUG DELIVERY TO OCULAR TISSUE

(71) Applicant: Regeneron Pharmaceuticals, Inc., Tarrytown, NY (US)

(72) Inventors: Bibiana IGLESIAS, Hudson, NY (US); Carmelo ROMANO, Tarrytown, NY (US)

(73) Assignee: Regeneron Pharmaceuticals, Inc., Tarrytown, NY (US)

(21) Appl. No.: 18/336,148

(22) Filed: Jun. 16, 2023

Related U.S. Application Data
(60) Provisional application No. 63/366,537, filed on Jun. 17, 2022.

(51) Int. Cl. A61M 5/32; A61M 37/00

(52) U.S. Cl. CPC A61M 5/3232; A61M 37/0015; A61M 2205/50; A61M 2205/3331

(57) ABSTRACT

Disclosed are devices and methods for facilitating directed delivery of a medicament into a human organ of a patient. An apparatus to facilitate directed delivery of a medicament into a human organ of a patient may include: a needle with a sharp distalmost tip; a needle hub connected to a proximal end of the needle; and an adaptor surrounding at least a portion of the needle; wherein the sharp distalmost tip may be configured to move from a retracted position in which the sharp distalmost tip is within the adaptor to a deployed position in which the sharp distalmost tip protrudes from the adaptor.

### CROSS-REFERENCE TO RELATED APPLICATION

This application claims priority to U.S. Provisional Patent Application No. 63/366,537, filed on Jun. 17, 2022 which is hereby incorporated by reference in its entirety.

### TECHNICAL FIELD

Various aspects of the present disclosure relate generally to delivering drugs to ocular tissue. More specifically, the present disclosure relates to instruments and related methods for delivering drugs to, e.g., the suprachoroidal space of an eye.

### INTRODUCTION

Eye conditions and diseases lead to optic nerve damage and visual field loss. Medications, laser surgery, and/or incisional surgery are interventions that may be employed to help lower intraocular pressure, save the subject's existing vision, and delay further progression of the condition and/or disease. With respect to incisional surgery, instruments for performing surgical procedures, devices for delivery drug therapies, and methods made possible by such instruments, are highly sought after to provide improved outcomes for users and subjects.

### SUMMARY OF THE DISCLOSURE

According to an aspect of the disclosure, a medicament delivery device apparatus may include: a needle with a sharp distalmost tip; a needle hub connected to a proximal end of the needle; and an adaptor surrounding at least a portion of the needle; wherein the sharp distalmost tip may be configured to move from a retracted position in which the sharp distalmost tip is within the adaptor to a deployed position in which the sharp distalmost tip protrudes from the adaptor.

Various embodiments of the apparatus may include one or more of the following aspects: a user-actuated mechanism configured to selectively move the sharp distalmost tip between the retracted position and the deployed position; a biasing member configured to urge the sharp distalmost tip toward the retracted position; one or more sensors; and a microprocessor configured to receive signals from the one or more sensors and, based on the signals, cause the sharp distalmost tip to move from the retracted position to the deployed position; and the one or more sensors may include a capacitance and/or pressure sensors positioned on the adaptor. Sensors positioned on the adaptor may also be used to determine the distance that the distalmost tip should move forward.

According to another aspect of the disclosure, a medicament delivery device apparatus may include: a needle with a sharp distalmost tip; a needle hub connected to a proximal end of the needle; an adaptor surrounding a portion of the needle; one or more sensors; and a microprocessor configured to receive signals from the one or more sensors and, based on the signals, determine a position of the sharp distalmost tip or adaptor relative to the human organ.

Various embodiments of the apparatus may include one or more of the following aspects: the one or more sensors may include a capacitance sensor positioned on the adaptor; the one or more sensors may include a plurality of pressure sensors positioned on the adaptor; a microneedle; the needle and the microneedle may be electrically connected via a low voltage circuit; the one or more sensors may include a first electrode positioned on at least one of the microneedle and the adaptor and a second electrode positioned near distalmost tip; the one or more sensors may include a level configured to determine an angular position of the needle; the apparatus may include a mechanism configured to move the sharp distalmost tip from a retracted position in which the sharp distalmost tip is positioned within the adaptor to a deployed position; the microprocessor may be configured to cause, in response to determining the position of the sharp distalmost tip or adaptor, the mechanism to move the sharp distalmost tip from the retracted position to the deployed position; the microprocessor may be configured to determine, based on the signals from the one or more sensors, that the sharp distalmost tip or adaptor has been moved out of contact with the human organ and cause, in response to determining that the sharp distalmost tip or adaptor has been moved out of contact with the human organ, the mechanism to move the sharp distalmost tip from the deployed position to the retracted position; the one or more sensors may include a sensor configured to detect an angular position of the needle relative to a tangent of the human organ and the microprocessor may be further configured to determine that the angular position of the needle is a predetermined angular position; the microprocessor may be configured to cause, in response to determining that the angular position of the needle is a predetermined angular position, the mechanism to move the sharp distalmost tip from the retracted position to the deployed position; the microprocessor may be configured to cause, in response to determining that the angular position of the needle is a predetermined angular position, one or more visual, audible, or tactile indications to be emitted; the microprocessor is may be configured to determine that a current of the low voltage circuit exceeds a predetermined current and cause, in response to determining that the current of the low voltage circuit exceeds the predetermined current, one or more visual, audible, or tactile indications to be emitted; the apparatus may include a first electrode positioned adjacent the sharp distalmost tip and a second electrode; the microprocessor may be configured to determine, based on a conductivity between the first electrode and second electrode, a position of the sharp distalmost tip and cause, in response to determining position of the sharp distalmost tip, one or more visual, audible, or tactile indications to be emitted.

In still another aspect of the disclosure, a kit may include a needle with a sharp distalmost tip; a container enclosing an ophthalmic drug; and an adaptor configured to be coupled to the needle such that the sharp distalmost tip is moveable from a retracted position in which the sharp distalmost tip is positioned within the adaptor to a deployed position in which the sharp distalmost tip protrudes from the adaptor.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

Moreover, there are several embodiments described and illustrated herein. The present disclosure is neither limited to any single aspect or embodiment thereof, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s).

FIG. 1 is a perspective view of an exemplary instrument, according to an embodiment of the present disclosure.

FIG. 2 is a cross-sectional view of an exemplary instrument, according to an embodiment of the present disclosure.

FIG. 3 depicts an exemplary instrument treating ocular tissue, according to an embodiment of the present disclosure.

FIG. 4 is a cross-sectional view of an exemplary instrument, according to a further embodiment of the present disclosure.

FIG. 5 is a perspective view of an exemplary instrument, according to another embodiment of the present disclosure.

FIGS. 6A and 6B are perspective views of an exemplary instrument, according to yet another embodiment of the present disclosure.

FIG. 7 is a perspective view of an exemplary instrument, according to an embodiment of the present disclosure.

FIG. 8 depicts an exemplary instrument treating ocular tissue, according to another embodiment of the present disclosure.

FIG. 9 depicts an exemplary instrument treating ocular tissue, according to a further embodiment of the present disclosure.

FIG. 10 depicts an exemplary instrument treating ocular tissue, according to an embodiment of the present disclosure.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art would appreciate that the side views are not drawn to scale and should not be viewed as representing proportional relationships between different components. The side views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. The term “distal” refers to a portion farthest away from a user when introducing a device into a subject. By contrast, the term “proximal” refers to a portion closest to the user when placing the device into the subject. In the discussion that follows, relative terms such as “about,” “substantially,” “approximately,” etc. are used to indicate a possible variation of ±10% in a stated numeric value.

Aspects of the disclosure relate to, among other things, instruments and methods for delivering drugs to ocular tissues. Each of the aspects disclosed herein may include one or more of the features described in connection with any of the other disclosed aspects. It may be understood that both the foregoing general description and the following detailed description are exemplary and explanatory only and are not restrictive of any claimed inventions.

While this disclosure describes certain instruments and methods, additional descriptions relevant to the instruments and methods described herein may be found in U.S. application Ser. No. 17/444,897, published as US 2022/0047420 A1, the entirety of which is incorporated herein by reference.

The suprachoroidal space (SCS) is a potential space between the sclera and choroid that traverses the circumference of the posterior segment of the eye. The SCS is a useful site for drug delivery because it targets the choroid, retinal pigment epithelium, and/or retina with high bioavailability, while maintaining low levels elsewhere in the eye. Under normal physiological conditions, primarily due to intraocular pressure (IOP), the SCS is primarily in a collapsed state. The SCS plays a role in maintaining IOP via uveoscleral outflow, which is an alternative drainage route for the aqueous humor, and is a natural flow path from the front to the back of the eye. Due to its role in maintaining IOP, the SCS has the potential to expand and contract in response to the presence of fluid. The SCS may expand to accommodate different volumes, for example, up to about 3.0 mm, depending on injection volumes. Injecting high volumes of drugs may have adverse effects, for example, elevated IOP, retinal elevation, choroidal hemorrhage away from needle entry, and choroidal edema and potential choroidal detachment; backflow from needle entry; and reflux of fluid which may cause subconjunctival hemorrhage. Additionally, high volumes of fluid may not be injected into the eye until a needle of an injection device has fully penetrated the sclera.

To expand the SCS, e.g., by separating the sclera and choroid mechanically and breaking down fibers holding the sclera and choroid together, instruments may be inserted through the sclera and placed at the correct depth between the sclera and choroid layers, such that optimal volumes of fluids, e.g., drugs or other suitable therapeutic agents, may be injected into the SCS. Any drugs inserted into the SCS may allow for direct drug delivery to the posterior section of the eye to specifically target, e.g., the retina and/or macula. The SCS may also be a useful destination for slow-release formulations such as depot drugs. For example, a depot drug inserted into the SCS may be useful for treating portions in the rear of the eye, such as the retina, retinal pigment epithelium (RPE), choroid, or other portions. From within the SCS, the depot drug may effectively target portions of the rear of the eye without impinging on a visual axis of the eye. Instruments and methods for insertion and injection into the eye may only allow for extension into a certain depth of the ocular layers. For example, under physiological conditions, the sclera layer ranges from about 300 μm to about 1100 μm, the SCS has a thickness of about 35 μm, and the choroid layer ranges from about 50 μm to about 300 μm. Depth of insertion of an instrument for drug delivery into the ocular layers may range from about 0.5 mm to about 1.1 mm. However, such a depth of insertion may penetrate and/or impact additional layers of the ocular tissue, e.g., the choroid, retinal pigment epithelium (RPE), and retina. Penetration of such layers should be minimized as much as possible, such that the desired drug may be directed into the targeted area of the eye via a minimally invasive procedure. For example, injection procedures may be performed as an outpatient procedure.

Instruments and methods discussed in the present disclosure address the disadvantages described above, and may increase the ability of the SCS to hold and diffuse optimal volumes of drugs, for example, approximately 50 μL to approximately 500 μL.

The example embodiments described herein may be used in the treatment of a variety of conditions, including ocular conditions. For example, embodiments of the present disclosure may be used in the treatment of refractive errors, macular degeneration, cataracts, retinopathy, retinal detachments, glaucoma, amblyopia, strabismus, any other ocular condition, or any other condition suitable for treatment via tissue in the eye.

The description herein and examples are illustrative and are not intended to be restrictive. One of ordinary skill in the art may make numerous modifications and/or changes without departing from the general scope of the invention. For example, and as has been referenced, aspects of above-described embodiments may be used in any suitable combination with each other. Additionally, portions of the above-described embodiments may be removed without departing from the scope of the invention. In addition, modifications may be made to adapt a particular situation or aspect to the teachings of the various embodiments without departing from their scope. Many other embodiments will also be apparent to those of skill in the art upon reviewing the above description.

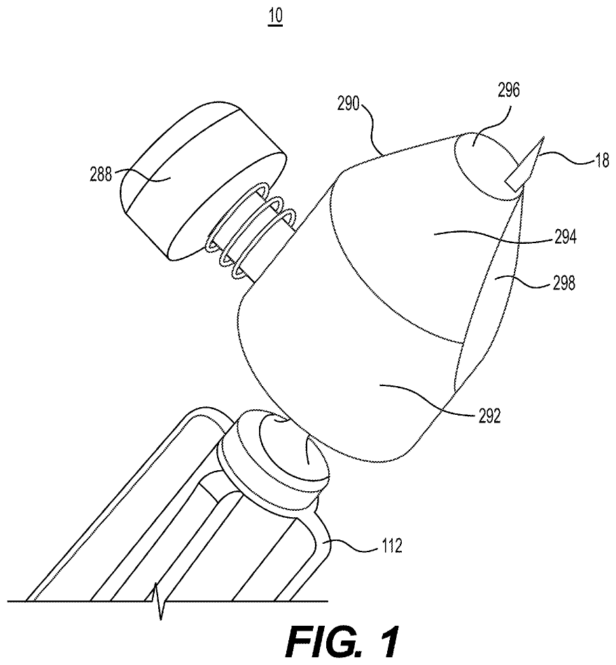

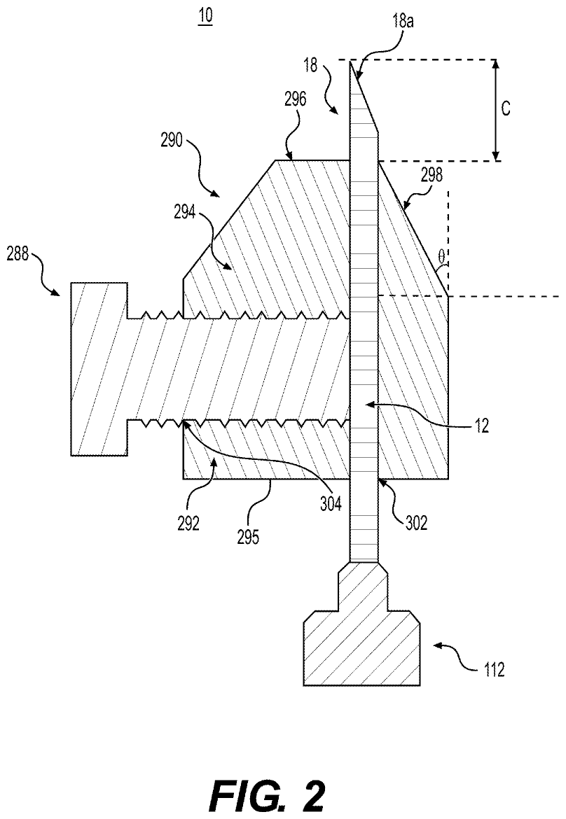

FIGS. 1 and 2 depict an example of an instrument 10 in accordance with the present disclosure. Instrument 10 may include a needle 12 having a passage therethrough configured to serve as a conduit for a medicament. Needle 12 may further include a distalmost tip 18. Distalmost tip 18 may be a sharp tip or needle configured to penetrate a tissue layer of the eye, e.g., a sclera. Needle 12 may be coupled to a needle hub 112, which may further be coupled to a container (not shown). A medicament may be contained within needle 12, needle hub 112, the container, or any combination thereof. In some examples, needle 12 may be a staked needle. In other examples, needle hub 112 may be disposed between the container and needle 12.

Components of instrument 10 may be made of any suitable metal, polymer, and/or combination of metals and/or polymers. Exemplary metallic materials may include stainless steel, nitinol, titanium, and/or alloys of these metals. Exemplary polymeric materials may include polyetheretherketone (PEEK), polyimide, and polyethersulfone (PES). In some examples, components of instrument 10 may be made of a rigid material, semi-rigid material, or flexible material, wherein such material may be expandable and/or may allow for various configurations as discussed herein. Materials of instrument 10 may be any biocompatible material that may be sterilized.

Instrument 10 may further include an adaptor 290. Adaptor 290 may be a component configured to surround a shaft of needle 12. Adaptor 290 may be positioned toward distalmost tip 18 relative to needle hub 112 to which needle 12 may be connected. Adaptor 290 may include an intermediate surface 292 which defines a substantially cylindrical portion of adaptor 290. When adaptor 290 is positioned to surround a portion of needle 12, a longitudinal axis of the substantially cylindrical portion of adaptor 290 may extend parallel to a longitudinal axis of needle 12. For purposes of this disclosure, the longitudinal axis of the substantially cylindrical portion should be understood to be a longitudinal axis of adaptor 290.

Adjacent to intermediate surface 292, adaptor 290 may include an angled distal surface 294 disposed toward a distal end of adaptor 290 relative to intermediate surface 292. Angled distal surface 294 may define a substantially frustoconical portion or a partially frustoconical portion of adaptor 290. Angled distal surface 294 may be oriented at an angle ranging from about 30 degrees to about 60 degrees relative to the longitudinal axis of adaptor 290, at an angle ranging from about 40 degrees to about 50 degrees relative to the longitudinal axis of adaptor 290, or at about a 45-degree angle relative to the longitudinal axis of adaptor 290, for example.

Adaptor 290 may further include an outermost slanted surface 298. Outermost slanted surface 298 may be a planar surface adjacent to intermediate surface 292 and/or angled distal surface 294. Alternatively, outermost slanted surface 298 may be a convex surface configured to be placed against and mate with a sclera of a patient's eye. As shown in FIG. 2, outermost slanted surface 298 may be oriented at an angle θ relative to the longitudinal axis of adaptor 290. Angle θ may range from about 25 degrees to about 75 degrees relative to the longitudinal axis of adaptor 290, from about 40 degrees to about 65 degrees relative to the longitudinal axis of the adaptor 290, or from about 30 degrees to about 60 degrees relative to the longitudinal axis of adaptor 290. In an exemplary embodiment, the angle θ may be about 45 degrees relative to the longitudinal axis of adaptor 290.

Outermost slanted surface 298 may be configured in various manners for contact with a sclera of a patient's eye. For example, outermost slanted surface 298 may be smooth or polished to minimize abrasion of the sclera. Alternatively, outermost slanted surface 298 may be rough to minimize movement of adaptor 290 relative to the sclera. In some embodiments, outermost slanted surface 298 may include geometric features, such as protruding dimples, indented dimples, waves, other geometric features, or any combination thereof. Additionally, a coating may be applied to outermost slanted surface 298. The coating may be therapeutic, antibacterial, and/or sterilizing. In some embodiments, a topical anesthetic may be applied as a coating to outermost slanted surface 298. As another example, outermost slanted surface 298 may be formed by overmolding a material on adaptor 290. The overmolded material may be selected, for example, based on its surface properties (e.g., rough, smooth, etc.) or its suitability for surface finishing, such as polishing. Outermost slanted surface 298 may further incorporate various combinations of the aforementioned features, such as a polished surface with geometric features, a rough surface with geometric features, an overmolded material with a coating, etc. While exemplary combinations of features have been described herein, these combinations are not intended to be limiting and other combinations are contemplated.

Adaptor 290 may include visual indications of a position of adaptor 290 and/or of outermost slanted surface 298. For example, outermost slanted surface 298 may be colored differently than other surfaces of adaptor 290 to distinguish outermost slanted surface 298 from the other surfaces. Adaptor 290 may also include visible markings to indicate a position adaptor 290 and/or of outermost slanted surface 298. Such visible markings may include markings of contrasting color, textured markings, or the like on outermost slanted surface 298 and/or on other surfaces of adaptor 290. The visible markings may be applied to adaptor 290 using silk-screening, overmolding, etching, or various other suitable techniques. The visible markings may be of any geometric shape, including circles, ovals, polygons, irregular shapes, or any combination thereof.

Adaptor 290 may include a proximal surface 295 and a distal surface 296. Proximal surface 295 may be a substantially circular surface adjacent to intermediate surface 292 and existing in a plane perpendicular to the longitudinal axis of adaptor 290. Distal surface 296 may also be a substantially circular surface. Distal surface 296 may be adjacent to angled distal surface 294 and exist in a separate plane perpendicular to the longitudinal axis of adaptor 290. Accordingly, proximal surface 295 may be parallel to distal surface 296.

Adaptor 290 may include a needle bore 302 in which needle 12 may be positioned. Needle bore 302 may extend parallel or substantially parallel to the longitudinal axis of adaptor 290. When positioned in needle bore 302, needle 12 may intersect each of proximal surface 295 and distal surface 296. When positioned in the needle bore, distalmost tip 18 of needle 12 may extend a distance C from distal surface 296. A length of distance C may be such that a bevel 18a of distalmost tip 18 may extend from distal surface 296. The length of distance C may further be such that a portion of a shaft of needle 12 proximal to distalmost tip 18 may extend from distal surface 296. Distance C may be, for example, between 200 μm and 1200 μm, between 400 μm and 1000 μm, between 600 μm and 800 μm, or about 700 μm. In some implementations, bevel 18a and outermost slanted surface 298 may be oriented at the same angle relative to the longitudinal axis of the adaptor 290.

Adaptor 290 may be selectively translatable relative to needle 12 along the longitudinal axis of needle 12. Translation of adaptor 290 may be desirable to adjust the distance C, for example. In some embodiments, the adaptor 290 may be fastened to needle 12. Adaptor 290 may be connected to needle 12 by any suitable means, including by a screw, a fastener, a nut, a bolt, or adhesive. As an example, and as shown in FIGS. 1 and 2, adaptor 290 may be fastened to needle 12 using a screw 288. Screw 288 may be inserted into a threaded bore 304 within adaptor 290. When tightened, screw 288 may exert a force on needle 12 perpendicular to the longitudinal axis of needle 12. The force may result in friction in a longitudinal direction between needle 12 and screw 288 as well as between needle 12 and needle bore 302, thereby preventing adaptor 290 from translating relative to needle 12. If the user wishes to adjust the distance C, e.g., to extend a distance of distalmost tip 18 from distal surface 296, the user may loosen the bolt to thereby allow translation of adaptor 290 relative to needle 12.

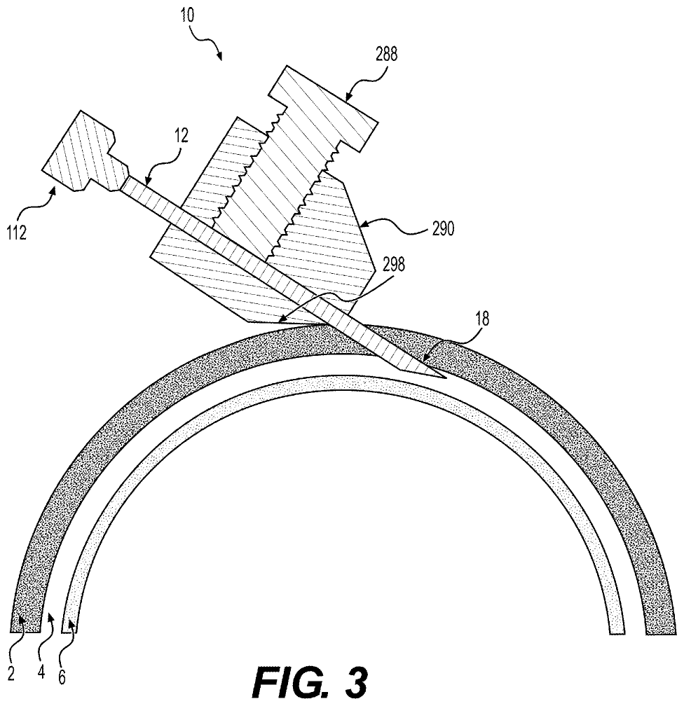

An exemplary use case for instrument 10 is depicted in FIG. 3, in which instrument 10 is shown relative to layers of the eye, i.e., sclera 2, SCS 4, and choroid 6. As shown, adaptor 290 may be used to guide a trajectory of distalmost tip 18 of needle 12 through sclera 2 into SCS 4. To inject a medicament into SCS 4, a user may, for example, penetrate sclera 2 with distalmost tip 18 and insert needle 12 through sclera 2. The user may angle needle 12 such that outermost slanted surface 298 is oriented parallel to a plane tangent to an outer surface of sclera 2. The user may then continue to insert needle 12 until the outermost slanted surface 298 contacts the surface of sclera 2. In an exemplary method in which outermost slanted surface 298 is a planar surface, the user may insert needle 12 until outermost slanted surface 298 is tangent with the surface of sclera 2. In an exemplary method in which outermost slanted surface 298 is a convex surface, the user may insert needle 12 until outermost slanted surface 298 mates with the surface of sclera 2. When outermost slanted surface 298 contacts sclera 2, needle 12 may be prevented from being inserted further and may be prevented from potentially penetrating choroid 6.

In some implementations, the user may be able to adjust the distance C to a desired length by translating adaptor 290 along needle 12. When the user has adjusted distance C and/or angle θ as desired, the user may use adaptor 290 to guide a trajectory of needle 12 into SCS 4 such that it penetrates sclera 2 at a substantially predetermined depth. Thereby, the user may be able to inject the medicament into the suprachoroidal space 4 with relative accuracy without penetrating choroid 6.

As shown in FIGS. 1-3, adaptor 290 may be positioned about needle 12. Adaptor 290 may alternatively be attached to either or both of hub 112 and a medicament container (e.g., a syringe) connected to needle 12. In some embodiments, adaptor 290 may be spring-loaded such that a spring urges adaptor 290 toward distalmost tip 18. In use, the user may place adaptor 290 against the patient's sclera and exert a force sufficient to depress the spring, thereby exposing needle 12. The spring may be configured to control a depth of penetration of needle 12 into the patient's eye.

Adaptor 290 may be made of any suitable metal, polymer, and/or combination of metals and/or polymers. Exemplary metallic materials may include stainless steel, nitinol, titanium, and/or alloys of these metals. Exemplary polymeric materials may include polyetheretherketone (PEEK), polyimide, and polyethersulfone (PES). In some examples, adaptor 290 may be made of a rigid material, semi-rigid material, or flexible material. Adaptor 290 may further be formed of any biocompatible material that may be sterilized. In some examples, adaptor 290 may be made of a transparent material to permit easier identification of, and/or navigation relative to, blood vessels in a patient's eye.

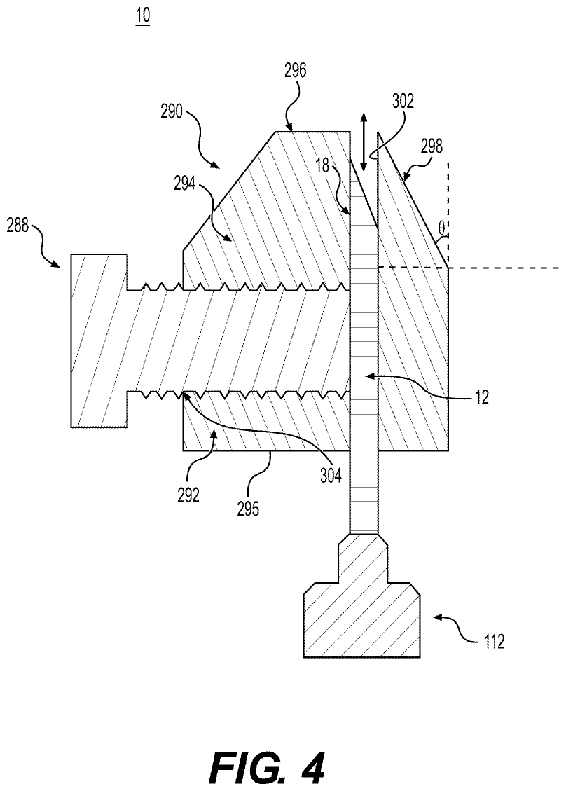

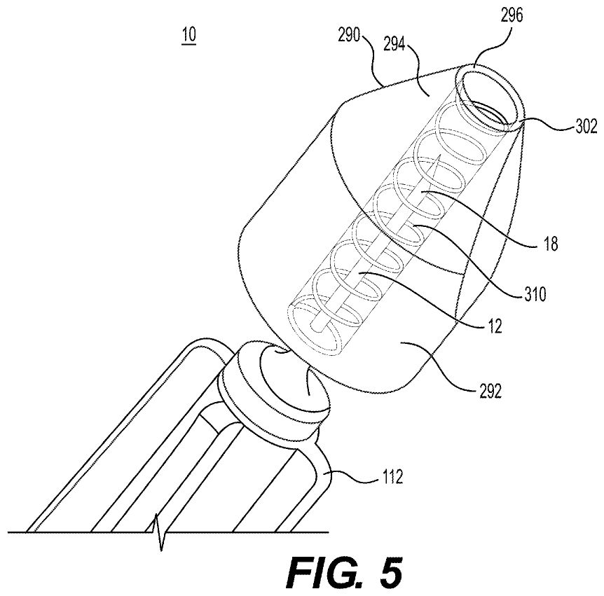

In some embodiments, needle 12 and/or distalmost tip 18 may be retractable. Specifically, distalmost tip 18 may be moveable between a retracted position, in which distalmost tip 18 is positioned within adaptor 290, and a deployed position, in which distalmost tip 18 protrudes from adaptor 290. For example, as shown in FIGS. 4 and 5, prior to use of instrument 10, distalmost tip 18 may be in the retracted position within needle bore 302 of adaptor 290 to prevent inadvertent insertion of distalmost tip 18 or intended injury thereby. During an injection, distalmost tip 18 may be moved to the deployed position, which may be a position similar to the position shown in FIGS. 1, 2, and 3, in which distalmost tip 18 protrudes at least partially out of needle bore 302 past distal surface 296.

Instrument 10 may include a mechanism to move distalmost tip 18 from the retracted position to the deployed position. The mechanism may be of any suitable type, such as a manually powered mechanism, an electrically powered mechanism, a motor driven mechanism, a spring driven mechanism, a compressed gas mechanism, or the like, or any combination thereof. In some embodiments, the mechanism may be user-actuated such that the user may selectively deploy and retract distalmost tip 18. In other embodiments, the deployment of distalmost tip 18 may be user-actuated, but the retraction of distalmost tip 18 may occur automatically at the end of a dose delivery event. In one example, as shown in FIG. 5, the mechanism may include an elastic member 310. Elastic member 310 may be a spring, for example, and may bias needle 12 and/or distalmost tip 18 toward the retracted position. When the user wishes to move distalmost tip 18 from the retracted position to the deployed position, the user may cause elastic member 310 to be depressed, thereby allowing distalmost tip 18 to protrude past distal surface 296. The user may cause elastic member 310 to be depressed using a button, switch, slide, or any other suitable mechanism. In some embodiments, distalmost tip 18 may be configured to remain in the deployed position once moved from the retracted position until the user takes further action to move distalmost tip 18 back to the retracted position. In some embodiments, distalmost tip 18 may be configured to move to the retracted position unless the user continues to actively depress elastic member 310.

In some embodiments, the mechanism that moves distalmost tip 18 from the retracted position to the deployed position may be responsive to signals transmitted by one or more sensors. For example, instrument 10 may include a microprocessor. The microprocessor may be configured to receive signals from one or more sensors and may further be configured to control the mechanism that moves distalmost tip 18 from the retracted position to the deployed position.

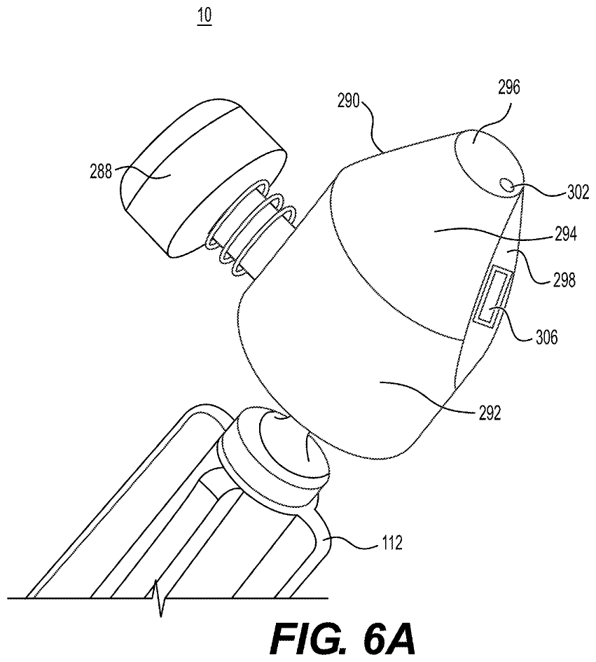

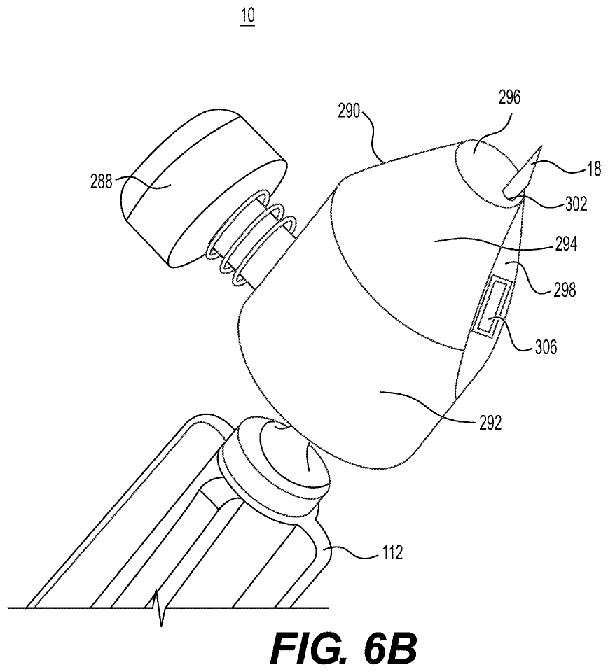

In some embodiments, as shown in FIGS. 6A and 6B, instrument 10 may include a capacitance sensor 306. Capacitance sensor 306 may be positioned on outermost slanted surface 298 of adaptor 290. When capacitance sensor 306 is placed in contact with a sclera of an eye, for example, capacitance sensor 306 may be configured to transmit a signal indicative of contact with the sclera to the microprocessor. In response to receiving the signal, the microprocessor may cause the mechanism to move distalmost tip 18 from the retracted position to the deployed position. In some embodiments, capacitance sensor 306 may be configured to transmit a signal indicative of scleral and choroidal thickness to the microprocessor. In response to the signal, the microprocessor may calculate a distance that the distalmost tip 18 may safely travel forward into the eye. The microprocessor may then cause the mechanism to move distalmost tip 18 forward the calculated distance.

In practice, distalmost tip 18 may initially be in the retracted position prior to an injection, as shown in FIG. 6A. When the user is ready to perform an injection, the user may place outermost slanted surface 298 against the sclera of an eye. Upon placement of outermost slanted surface 298 against the sclera, capacitance sensor 306 may contact the sclera and detect the capacitance thereof. Upon detection of the capacitance of the sclera, capacitance sensor 306 may transmit a signal indicative of contact with the sclera to the microprocessor. In response to receiving the signal, the microprocessor may cause distalmost tip 18 to move from the retracted position to the deployed position, as shown in FIG. 6B. Due to the position of instrument 10 relative to the eye when capacitance sensor 306 detects the sclera, the distalmost tip 18 may penetrate the sclera when moving from the retracted position to the deployed position.

In some embodiments, capacitance sensor 306 may continue to transmit signals to the microprocessor when distalmost tip 18 is in the deployed position. As long as capacitance sensor 306 continues to transmit signals indicating that it remains in contact with the sclera, the mechanism may maintain distalmost tip 18 in the deployed position. If, on the other hand, capacitance sensor 306 is moved out of contact with the sclera, a signal indicating that capacitance sensor 306 is no longer in contact with the sclera may be transmitted to the microprocessor. In response, the microprocessor may cause the mechanism to move distalmost tip 18 to the retracted position.

In some embodiments, the microprocessor may be configured to determine that a drug has been completely administered from instrument 10 or otherwise that a desired amount of a drug has been administered from instrument 10. In response to a determination that the drug has been completely administered or that a desired amount has been administered, the microprocessor may cause the mechanism to move distalmost tip 18 to the retracted position. The microprocessor may initiate such retraction while capacitance sensor 306 remains in contact with the sclera to ensure safe removal of instrument 10 from the patient.

Alternatively, in embodiments in which the mechanism is manually operated, a signal from the capacitance sensor 306 indicative of contact with the sclera may cause one or more visual, audible, or tactile indications to be communicated to the user. For example, upon contact with the sclera, a light on instrument 10 may be illuminated, indicating to the user that instrument 10 is in a suitable position for injection. In another example, a sound may be emitted from instrument 10, indicating to the user that instrument 10 is in a suitable position for injection. In another example, instrument 10 may vibrate, indicating to the user that instrument 10 is in a suitable position for injection. Though examples of visual, audible, and tactile feedback are provided, it should be understood that any suitable indication may be provided to alert the user of a positioning of instrument 10.

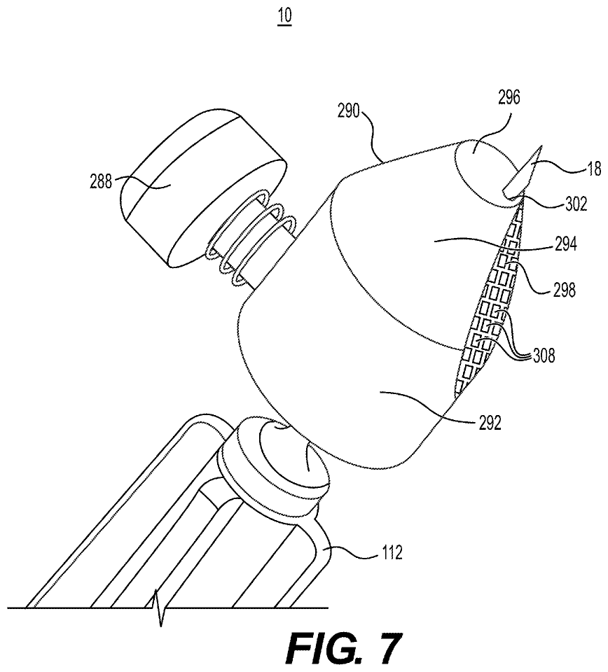

In some embodiments, as shown in FIG. 7, instrument 10 may include one or more pressure sensors 308 (in addition to or as an alternative to capacitance sensor 306). Similar to capacitance sensor 306, pressure sensors 308 may be positioned on outermost slanted surface 298 of adaptor 290. When pressure sensors 308 are placed in contact with a sclera of an eye, for example, each of the pressure sensors 308 may be configured to transmit a signal indicative of a pressure asserted by the sclera. In response to receiving signals from the pressure sensors 308 indicative of contact with the sclera, the microprocessor may cause the mechanism to move distalmost tip 18 from the retracted position (not shown) to the deployed position (shown in FIG. 7). In some embodiments, the microprocessor may cause the mechanism to move distalmost tip 18 from the retracted position to the deployed position in response signals indicating a uniform or near-uniform pressure applied across the pressure sensors 308. An indication of a uniform or near-uniform pressure applied across the pressure sensors 308 may signify that outermost slanted surface 298 is uniformly pressed against the sclera, as opposed to positioned at an angle, positioned unfirmly, or the like.

In practice, distalmost tip 18 may initially be in the retracted position prior to an injection. When ready to perform an injection, the user may place outermost slanted surface 298 against the sclera of an eye. Upon placement of outermost slanted surface 298 against the sclera, one or more pressure sensors 308 may contact the sclera and detect the pressure applied to them. Upon detection of pressure indicating contact with the sclera, the one or more pressure sensors 308 may transmit signals indicative of contact with the sclera to the microprocessor. In response to receiving the signals, the microprocessor may cause distalmost tip 18 to move from the retracted position to the deployed position, as shown in FIG. 7. Due to the position of instrument 10 relative to the eye when pressure sensors 308 detect pressures applied by the sclera, the distalmost tip 18 may penetrate the sclera when moving from the retracted position to the deployed position.

In some embodiments, pressure sensors 308 may continue to transmit signals to the microprocessor when distalmost tip 18 is in the deployed position. As long as pressure sensors 308 continue to transmit signals indicative of the sensors being in contact with the sclera, the mechanism may maintain distalmost tip 18 in the deployed position. If, on the other hand, pressure sensors 308 have moved out of contact with the sclera, signals indicating that pressure sensors 308 are no longer in contact with the sclera may be transmitted to the microprocessor. In response, the microprocessor may cause the mechanism to move distalmost tip 18 to the retracted position.

In some embodiments, the microprocessor may be configured to determine that a drug has been completely administered from instrument 10 or otherwise that a desired amount of a drug has been administered from instrument 10. In response to a determination that the drug has been completely administered or that a desired amount has been administered, the microprocessor may cause the mechanism to move distalmost tip 18 to the retracted position. The microprocessor may initiate such retraction while pressure sensors 308 remain in contact with the sclera to ensure safe removal of instrument 10 from the patient.

Alternatively, in embodiments in which the mechanism is manually operated, signals from pressure sensors 308 indicative of contact with the sclera may cause one or more visual, audible, or tactile indications to be communicated to the user, as described herein previously.

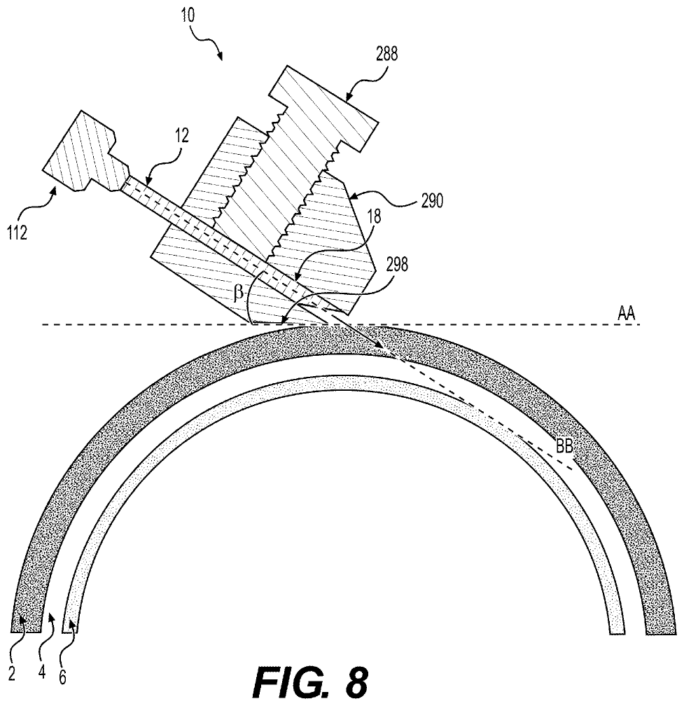

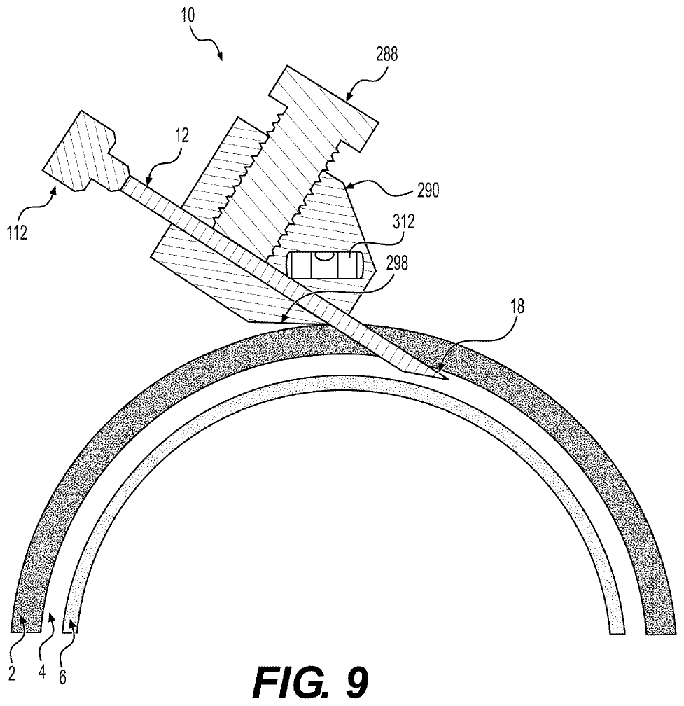

In some embodiments, as shown in FIGS. 8 and 9, instrument 10 may be configured to detect and/or operate according to its angular position. For example, as shown in FIG. 8, instrument 10 may include one or more sensors (e.g., positional or gyroscopic sensors) configured to detect an angle β of an axis BB extending through needle 12 relative to an axis AA extending tangent to sclera 2. The one or more sensors may include image sensors, gyroscopic sensors, accelerometers, combinations thereof, or any other suitable sensors. Each of the sensors may be configured to transmit signals to the microprocessor, which in turn may be configured to calculate angle β based on the signals. In response to a determination that angle β is a suitable angle for injection, the microprocessor may cause the mechanism to move distalmost tip 18 from the retracted position to the deployed position.

In another example, as shown in FIG. 9, instrument 10 may include a level 312 or any other suitable mechanical, electromechanical, or electrical position determining mechanism. In some embodiments, level 312 may be a bubble level, for instance. Level 312 may be angularly offset from needle 12, such that when level 312 is horizontal, needle 12 is at a desired angle relative to horizontal. In use, instrument 10 may be oriented such that level 312 is positioned horizontally (e.g., the bubble is centered). When level 312 is horizontal, needle 12 may be positioned at the desired angle for penetration into the SCS 4. In some embodiments, in response to being placed in a horizontal orientation, level 312 may transmit a signal to the microprocessor indicative of the horizontal orientation. In response to the signal, the microprocessor may cause the mechanism to move distalmost tip 18 from the retracted position to the deployed position.

In some embodiments, the microprocessor may be configured to determine that a drug has been completely administered from instrument 10 or otherwise that a desired amount of a drug has been administered from instrument 10. In response to a determination that the drug has been completely administered or that a desired amount has been administered, the microprocessor may cause the mechanism to move distalmost tip 18 to the retracted position.

The embodiments shown in FIGS. 8 and 9 may alternatively be manually operated. In such embodiments, signals from the sensors and/or level 312 may cause one or more visual, audible, or tactile indications to be communicated to the user, as described herein previously. Subsequently, a user may selectively deploy and retract needle 12 as desired or clinically necessary.

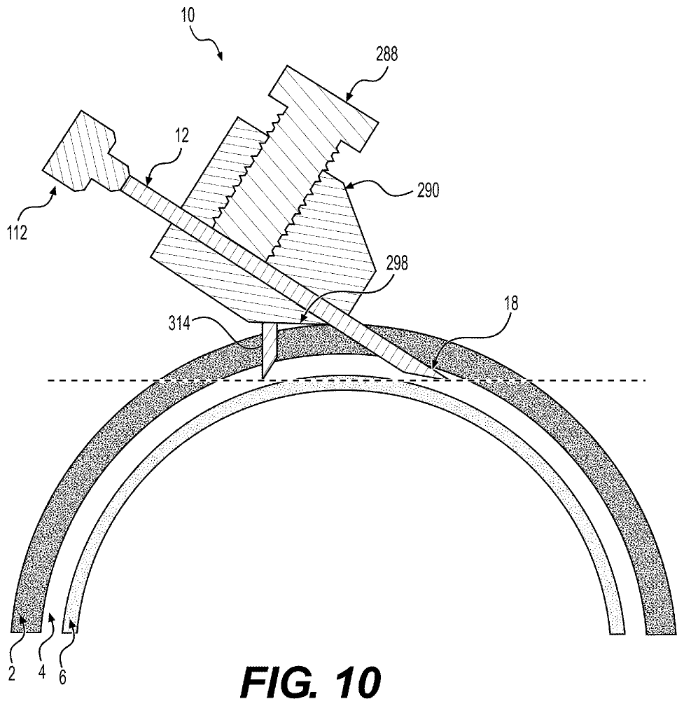

In some embodiments, instrument 10 may be configured to alert the user if distalmost tip 18 has been inserted too deeply into a subject's eye. In such an example, instrument 10 may include a microneedle 314 positioned thereon, as shown in FIG. 10. Microneedle 314 may be positioned in various locations on instrument 10, including on adaptor 290, on needle hub 112, along a shaft of needle 12, on a syringe, or in any other suitable location. In some embodiments, microneedle 314 may be coupled to, for example, outermost slanted surface 298. In such an embodiment, microneedle 314 and needle 12 may both be formed of conductive materials and may be electrically connected to each other on a low voltage circuit. Microneedle 314 may extend a fixed length from the remainder of instrument 10 and may be configured to be inserted to the outermost surface of choroid 6. If distalmost tip 18 is inserted through SCS 4 into choroid 6, an increased electric current may flow through the low voltage circuit. The increased electric current may be detected by the microprocessor and in response to detecting the increased current, the microprocessor may cause one or more visual, audible, or tactile indications to be communicated to the user. The one or more visual, audible, or tactile indications may alert the user that distalmost tip 18 has been inserted too deeply.

In some embodiments, microneedle 314 may be configured to be deployed from and retracted into instrument 10. For example, as described herein previously, capacitance sensor 306 may be configured to transmit a signal indicative of scleral and choroidal thickness to the microprocessor. In response to the signal, the microprocessor may calculate a distance that the microneedle 314 may safely travel to reach the outermost surface of choroid 6. The microprocessor may then cause a deployment mechanism to move microneedle 314 the calculated distance into the eye for insertion into the outermost surface of choroid 6.

In some embodiments, in addition to or in lieu of microneedle 314, instrument 10 may include an electrode. In some embodiments, the electrode may be positioned on microneedle 314 and in some embodiments the electrode may be positioned on outermost slanted surface 298. The electrode may be electrically connected to an electrode positioned near distalmost tip 18 on a low voltage circuit. Based on a detected conductivity between the electrodes, the microprocessor may determine whether the electrode on distalmost tip 18 is in contact with sclera 2, is positioned within SCS 4, or is in contact with choroid 6. The microprocessor may be configured to cause one or more visual, audible, or tactile indications to be communicated to the user, where the indications vary depending on the location of distalmost tip 18. The indications may alert the user as to whether distalmost tip 18 has been inserted to a desired depth within the eye (e.g., to the SCS), or whether distalmost tip 18 has been inserted either too shallowly or too deeply.

While instrument 10 is described herein and shown in the associated figures as including a needle 12 that extends through adaptor 290, it should be understood that such a needle is not necessarily required. For example, instrument 10 may instead include a microneedle positioned toward distal surface 296 that does not extend entirely through adaptor 290. In such an embodiment, adaptor 290 may include a fluid conduit therein that may be in fluid communication with the microneedle. Instrument 10 may be configured such that medicament flows through the fluid conduit to the microneedle and into a patient. Such a microneedle may be moveable, as described herein previously, from a retracted position within adaptor 290 to a deployed position in which the microneedle protrudes beyond distal surface 296.

It is to be understood that any dimensions of adaptor 290 perceived from the figures are not intended to be limited and indeed may vary. For example, a length of adaptor 290 (i.e., a distance between proximal surface 295 and distal surface 296) may vary to accommodate needles of different lengths. Also, a diameter of needle bore 302 may vary to accommodate needles having different diameters. Further, diameters of proximal surface 295 and/or distal surface 296 may vary.

As described herein, adaptor 290 may be useful for reducing human error in ocular injection procedures. In addition to being useful for injections into the suprachoroidal space, adaptor 290 may be useful for injections into other spaces in the eye, such as the subretinal space. Current methods for subretinal drug delivery may be invasive and may further require surgery. Surgical procedures for subretinal drug delivery may involve creating tears on the retinal surface and/or full vitrectomies in order to allow for a cannula to access the subretinal space. Alternatively, adaptor 290 may allow access to the subretinal space through the sclera, thereby decreasing the invasiveness of the procedure. Using eye imaging techniques such as optical coherence tomography (OCT) and/or ultrasound, an accurate distance between the surface of the sclera and the subretinal space may be calculated. A distance between distalmost tip 18 of needle 12 and distal surface 296 or outermost slanted surface 298 of adaptor 290 may be configured to match the distance between the sclera and the subretinal space. In such a configuration, adaptor 290 may prevent needle 12 from extending beyond the subretinal space into the vitreous. Outermost slanted surface 298 may also control an angle at which the subretinal injection is performed.

Adaptor 290 may be formed by any suitable manufacturing process, including but not limited to milling, CNC machining, polymer casting, rotational molding, vacuum forming, injection molding, extrusion, blow molding, or any combination thereof.

The various devices and components described herein may be provided in a kit for practicing one or more of the methods described herein. For example, a syringe, a needle, an adaptor, and an amount of ophthalmic drug may be provided in a blister pack. Each of the syringe, the needle, the adaptor, and the ophthalmic drug may be sealed within the blister pack after being sterilized. In some embodiments, a kit may include multiple adaptors. The multiple adaptors may have varying dimensions such that a user may select an adaptor best suited to a patient's anatomy and/or to control a penetration angle or depth of the needle. The multiple adaptors may also be formed from varying materials such that a user may choose an adaptor having an appropriate material for a particular procedure and/or patient. In some embodiments, the syringe may contain the ophthalmic drug. A nominal maximum fill volume of the syringe may be between about 0.5 mL and about 1.0 mL. In various methods described herein, a volume of the medicament, e.g., an ophthalmic drug, delivered to the patient may range from about 50 μL to about 500 μL.

Various drugs and formulations of drugs may be used with the embodiments of the present disclosure. As one example, embodiments described herein may be used to inject a drug in delayed-release pellet form. The drug may be released from the pellets when the pellets are hydrated, which may be achieved either by exposure of the pellets to fluids of the eye, by injecting a separate hydrating fluid, or by a combination of the foregoing. The separate hydrating fluid, such as saline, may be injected either before, after, or simultaneously with the pellets. As another example, embodiments described herein may be used to inject multiple substances in sequence. A first substance may be injected to expand a target space of the eye, such as the suprachoroidal space, and a second substance may subsequently be injected into the expanded suprachoroidal space. The first substance may be, for example, saline and the second substance may be, for example, a drug in a viscous gel form. As still another example, a sponge-like material may first be injected or inserted into a target space of the eye. The sponge-like material may be configured to release a drug over time. The sponge-like material may further be refilled or re-soaked with the drug by subsequent injections of the drug.

Drugs that may be used with embodiments of the present disclosure include: aflibercept (EYLEA®), triamcinolone acetonide suspension (ZUPRATA®), bevacizumab (AVASTIN®), and gene therapy drugs (including adeno-associated virus serotype 8 (AAV8) vectors for ocular gene transfer). Though examples are provided herein, these examples are not intended to be limiting and any suitable drug may be used with the embodiments of the present disclosure.

In embodiments of the present disclosure, needle 12 may be a first needle and the devices, apparatus, and/or kits disclosed herein may include a second needle. The first needle and the second needle may be interchangeable. Accordingly, needle 12 maybe be replaceable.

Listed below are further illustrative embodiments according to the present disclosure:

(1) A medicament delivery device apparatus comprising: a needle with a sharp distalmost tip; a needle hub connected to a proximal end of the needle; and an adaptor surrounding a portion of the needle; wherein the sharp distalmost tip is configured to move from a retracted position in which the sharp distalmost tip is within the adaptor to a deployed position in which the sharp distalmost tip protrudes from the adaptor.

(2) The apparatus of (1), further comprising a user-actuated mechanism configured to selectively move the sharp distalmost tip between the retracted position and the deployed position.

(3) The apparatus of (2), further comprising a biasing member configured to urge the sharp distalmost tip toward the retracted position.

(4) The apparatus of (1), further comprising one or more sensors; and a microprocessor configured to receive signals from the one or more sensors and, based on the signals, cause the sharp distalmost tip to move from the retracted position to the deployed position.

(5) The apparatus of (4), wherein the one or more sensors include a capacitance sensor positioned on the adaptor.

(6) The apparatus of (4), wherein the one or more sensors include a pressure sensor positioned on the adaptor.

(7) A medicament delivery device apparatus comprising: a needle with a sharp distalmost tip; a needle hub connected to a proximal end of the needle; an adaptor surrounding a portion of the needle; one or more sensors; and a microprocessor configured to receive signals from the one or more sensors and, based on the signals, determine a position of the sharp distalmost tip or adaptor relative to a human organ.

(8) The apparatus of (7), wherein the one or more sensors include a capacitance sensor positioned on the adaptor.

(9) The apparatus of (7), wherein the one or more sensors include a plurality of pressure sensors positioned on the adaptor.

(10) The apparatus of (7), further comprising a microneedle; wherein the needle and the microneedle are electrically connected via a low voltage circuit.

(11) The apparatus of (7), wherein the one or more sensors include a first electrode positioned on the adaptor and the microneedle and a second electrode positioned near distalmost tip.

(12) The apparatus of (7), wherein the one or more sensors include a level configured to determine an angular position of the needle and the adaptor.

(13) The apparatus of (7), further comprising: a mechanism configured to move the sharp distalmost tip from a retracted position in which the sharp distalmost tip is positioned within the adaptor to a deployed position; wherein the microprocessor is further configured to cause, in response to determining the position of the sharp distalmost tip or adaptor, the mechanism to move the sharp distalmost tip from the retracted position to the deployed position.

(14) The apparatus of (13), wherein the microprocessor is further configured to: determine, based on the signals from the one or more sensors, that the sharp distalmost tip or adaptor has been moved out of contact with the human organ; and cause, in response to determining that the sharp distalmost tip or adaptor has been moved out of contact with the human organ, the mechanism to move the sharp distalmost tip from the deployed position to the retracted position.

(15) The apparatus of (7), wherein the one or more sensors includes a sensor configured to detect an angular position of the needle relative to a tangent of the human organ; wherein the microprocessor is further configured to determine that the angular position of the needle is a predetermined angular position.

(16) The apparatus of (15), further comprising: a mechanism configured to move the sharp distalmost tip from a retracted position in which the sharp distalmost tip is positioned within the adaptor to a deployed position; wherein the microprocessor is further configured to cause, in response to determining that the angular position of the needle is a predetermined angular position, the mechanism to move the sharp distalmost tip from the retracted position to the deployed position.

(17) The apparatus of (15), wherein the microprocessor is further configured to cause, in response to determining that the angular position of the needle is a predetermined angular position, one or more visual, audible, or tactile indications to be emitted.

(18) The apparatus of (10), wherein the microprocessor is further configured to: determine that a current of the low voltage circuit exceeds a predetermined current; and cause, in response to determining that the current of the low voltage circuit exceeds the predetermined current, one or more visual, audible, or tactile indications to be emitted.

(19) The apparatus of (7), further comprising: a first electrode positioned adjacent the sharp distalmost tip and a second electrode; wherein the microprocessor is further configured to: determine, based on a conductivity between the first electrode and second electrode, a position of the sharp distalmost tip; and cause, in response to determining position of the sharp distalmost tip, one or more visual, audible, or tactile indications to be emitted.

(20) A kit, comprising: a needle with a sharp distalmost tip; a container enclosing an ophthalmic drug; and an adaptor configured to be coupled to the needle such that the sharp distalmost tip is moveable from a retracted position in which the sharp distalmost tip is positioned within the adaptor to a deployed position in which the sharp distalmost tip protrudes from the adaptor.

It will be apparent to those skilled in the art that various modifications and variations can be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

What is claimed is:

1. A medicament delivery device apparatus comprising:
a needle with a sharp distalmost tip;
a needle hub connected to a proximal end of the needle; and
an adaptor surrounding a portion of the needle;
wherein the sharp distalmost tip is configured to move from a retracted position in which the sharp distalmost tip is within the adaptor to a deployed position in which the sharp distalmost tip protrudes from the adaptor.

2. The apparatus of claim 1, further comprising:
a user-actuated mechanism configured to selectively move the sharp distalmost tip between the retracted position and the deployed position.

3. The apparatus of claim 2, further comprising:
a biasing member configured to urge the sharp distalmost tip toward the retracted position.

4. The apparatus of claim 1, further comprising:
one or more sensors; and
a microprocessor configured to receive signals from the one or more sensors and, based on the signals, cause the sharp distalmost tip to move from the retracted position to the deployed position.

5. The apparatus of claim 4, wherein the one or more sensors include a capacitance sensor positioned on the adaptor.

6. The apparatus of claim 4, wherein the one or more sensors include a pressure sensor positioned on the adaptor.

7. A medicament delivery device apparatus comprising:
a needle with a sharp distalmost tip;
a needle hub connected to a proximal end of the needle;
an adaptor surrounding a portion of the needle;
one or more sensors; and
a microprocessor configured to receive signals from the one or more sensors and, based on the signals, determine a position of the sharp distalmost tip or adaptor relative to a human organ.

8. The apparatus of claim 7, wherein the one or more sensors include a capacitance sensor positioned on the adaptor.

9. The apparatus of claim 7, wherein the one or more sensors include a plurality of pressure sensors positioned on the adaptor.

10. The apparatus of claim 7, further comprising:
a microneedle;
wherein the needle and the microneedle are electrically connected via a low voltage circuit.

11. The apparatus of claim 10, wherein the one or more sensors include a first electrode positioned on the adaptor and the microneedle and a second electrode positioned near the distalmost tip.

12. The apparatus of claim 7, wherein the one or more sensors include a level configured to determine an angular position of the needle and the adaptor.

13. The apparatus of claim 7, further comprising:
a mechanism configured to move the sharp distalmost tip from a retracted position in which the sharp distalmost tip is positioned within the adaptor to a deployed position;
wherein the microprocessor is further configured to cause, in response to determining the position of the sharp distalmost tip or adaptor, the mechanism to move the sharp distalmost tip from the retracted position to the deployed position.

14. The apparatus of claim 13, wherein the microprocessor is further configured to:
determine, based on the signals from the one or more sensors, that the sharp distalmost tip or adaptor has been moved out of contact with the human organ; and
cause, in response to determining that the sharp distalmost tip or adaptor has been moved out of contact with the human organ, the mechanism to move the sharp distalmost tip from the deployed position to the retracted position.

15. The apparatus of claim 7, wherein the one or more sensors includes a sensor configured to detect an angular position of the needle relative to a tangent of the human organ;
wherein the microprocessor is further configured to determine that the angular position of the needle is a predetermined angular position.

16. The apparatus of claim 15, further comprising:
a mechanism configured to move the sharp distalmost tip from a retracted position in which the sharp distalmost tip is positioned within the adaptor to a deployed position;
wherein the microprocessor is further configured to cause, in response to determining that the angular position of the needle is a predetermined angular position, the mechanism to move the sharp distalmost tip from the retracted position to the deployed position.

17. The apparatus of claim 15, wherein the microprocessor is further configured to cause, in response to determining that the angular position of the needle is a predetermined angular position, one or more visual, audible, or tactile indications to be emitted.

18. The apparatus of claim 10, wherein the microprocessor is further configured to:
determine that a current of the low voltage circuit exceeds a predetermined current; and
cause, in response to determining that the current of the low voltage circuit exceeds the predetermined current, one or more visual, audible, or tactile indications to be emitted.

19. The apparatus of claim 7, further comprising:
a first electrode positioned adjacent the sharp distalmost tip and a second electrode;
wherein the microprocessor is further configured to:
determine, based on a conductivity between the first electrode and second electrode, a position of the sharp distalmost tip; and
cause, in response to determining position of the sharp distalmost tip, one or more visual, audible, or tactile indications to be emitted.

20. A kit, comprising:
a needle with a sharp distalmost tip;
a container enclosing an ophthalmic drug; and
an adaptor configured to be coupled to the needle such that the sharp distalmost tip is moveable from a retracted position in which the sharp distalmost tip is positioned within the adaptor to a deployed position in which the sharp distalmost tip protrudes from the adaptor.
